(19) United States
(12) Patent Application Publication
(10) Pub. No.: US 2020/0000369 A1
(43) Pub. Date: Jan. 2, 2020

(54) UNOBTRUSIVE HEALTH ANALYSIS

(71) Applicant: KONINKLIJKE PHILIPS N.V., Eindhoven (NL)

(72) Inventors: CHRISTIAN ANDREAS TIEMANN, EINDHOVEN (NL); CORNELIS BERNARDUS ALOYSIUS WOUTERS, ECHT (NL)

(21) Appl. No.: 16/454,213

(22) Filed: Jun. 27, 2019

(30) Foreign Application Priority Data
Jun. 27, 2018 (EP) 18180023.6

(51) Int. Cl. A61B 5/08; A61B 5/097; G01N 33/497; A61B 5/083

(52) U.S. Cl. CPC A61B 5/082; A61B 5/097; A61B 5/4866; A61B 5/083; A61B 5/0816; G01N 33/497

(57) ABSTRACT

A method of estimating a ventilation rate in a room, the method comprising receiving a signal indicative of a concentration of at least one Volatile organic compound (VOC(t)) inside the room; processing the signal to detect at least one increase from an initial level (VOC(t0)) of the concentration of the at least one volatile organic compound; detect a corresponding maximum level of the concentration from the initial level; detecting when the concentration of the volatile organic compound returns towards the initial level or a steady state level (VOCss) from the corresponding maximum level; and determining the ventilation rate (k) based on the rate at which the concentration of the at least one Volatile organic compound returns towards the initial level or the steady state level from the corresponding maximum level.

### FIELD OF THE INVENTION

The present invention relates to the field of health analysis, in particular unobtrusive health analysis.

### BACKGROUND OF THE INVENTION

Exhaled breath analysis is becoming an increasingly important non-invasive diagnostic method that can be used in the evaluation of health status and disease types. Thousands of molecules are emitted into the air when we breathe. A breath sample is typically composed of inorganic gases (e.g., NO, CO₂, and CO), Volatile Organic Compounds (VOCs) (e.g., isoprene, ethane, pentane, and acetone), and other non-volatile substances (e.g., isoprostanes, peroxynitrite, cytokines, and nitrogen). FIG. 1 shows an overview of the most prominent VOCs present in human breath (Agapiou et al. Trace detection of endogenous human volatile organic compounds for search, rescue and emergency applications, Trends in Analytical Chemistry 66 (2015) 158-175). Detailed analysis of breath components can provide information about physiological processes that take place in the body, and hereby provide information of the current health status. For instance, a VOC of frequent occurrence is acetone which is a ketone body being produced by the liver from fatty acids during periods of low food intake. Acetone is also known to be elevated in diabetic patients. Furthermore, breath analyses have been successfully employed to identify conditions like lung disease, oxidative stress, gastrointestinal disease, and metabolic disorders.

Although breath analysis is non-invasive in nature, which is ideal in a hospital setting to replace certain blood tests, there are limitations to use it in a home setting, for instance for regular monitoring. Breath analysis could be perceived as obtrusive as it still requires a device to capture a breath sample (e.g., a mask or tube). This might not be comfortable and it is not very attractive to perform tests on a regular basis. Next to this, it might also be costly as such a device probably requires a disposable part for hygiene reasons.

### SUMMARY OF THE INVENTION

It is an object of one aspect of the present invention to provide an unobtrusive way to analyze health of the person by means of detecting certain compounds of interest and their release rate of these compounds of interest. These compounds of interest are released by a person in a room, for instance by means of breath, flatulence, through skin, etc. In various embodiments of the invention, the person may be a baby, an elderly or any other human being. The methods described below are best suited for a single person located in a room for a prolonged period of time, e.g., during sleep. It is another aspect of the invention to provide a method of estimating a ventilation rate of a room.

The invention proposes to estimate the rate of exhaled compounds of interest in an indirect way by measuring the concentration of these compounds in the air of the room the person is located in, and by estimating the ventilation rate of the room. The air concentration of a compound is determined by the inflow of the compound into the room (e.g., via human breathing) and the outflow of the compound to adjacent areas via doors and windows. The ventilation rate helps to determine the release rate of the compound of interest, which thereby helps to understand the amount of compound of interest being released in a period of time, which further then facilitates determining the health of the person.

In a first aspect of the present disclosure a method of estimating a ventilation rate in a room is provided. The method comprising:

receiving a signal indicative of a concentration of at least one Volatile Organic Compound (VOC(t)) inside the room, wherein the compound is released by at least one of exhalation, flatulence and release of the compound from the skin of a person in the room;

processing the signal to
detect at least one increase from an initial level (VOC(t0)) of the concentration of the at least one volatile organic compound;
detect a corresponding maximum level of the concentration from the initial level;
detect the return of the concentration of the volatile organic compound returns towards the initial level or a steady state level (VOCss) from the corresponding maximum level; and
determine the ventilation rate (k) based on the rate at which the the concentration of the at least one Volatile organic compound returns towards the initial level or the steady state level (VOCss) from the corresponding maximum level.

Advantageously a method of estimating a ventilation rate is provided which makes use of only a Volatile Organic Compound (VOC) sensor to understand the temporary increases of VOCs, mostly gases, in the room and subsequent diffusion, which is effected by the ventilation rate of the room. Since the VOCs used for determining ventilation are temporary in nature, such as VOCs released by flatulence, it is easy to distinguish the abrupt increases in the VOC signal as these VOCs also subside (diffuse) within a few minutes, due to ventilation.

In a further aspect of the present disclosure, the method further includes estimating release rate of a compound of interest, wherein the release rate is of the person, wherein estimating the release rate further comprises:
receiving a signal indicative of a first concentration of the compound of interest inside the room, wherein the compound of interest is being released by the person;
receiving a signal indicative of a second concentration of the compound of interest outside the room; and
calculating the release rate of the compound of interest based on the first concentration, second concentration and the ventilation rate.

Advantageously, the method steps above helps in determination of the release rate of the compound of interest in a non-obtrusive way. The person concerned is not disturbed in any sense and also is not asked to wear any mask or to breathe in a device to understand the release rate of certain compound of interest in his breath (for instance).

In another aspect of the present disclosure, an apparatus for estimating a ventilation rate in a room is provided, the apparatus includes:
a. a signal interface configured for receiving a signal indicative of a concentration of at least one Volatile organic compound inside the room, wherein the compound is released by at least one of exhalation, flatulence and release of the compound from the skin of a person in the room and wherein the signal is received from a sensor configured for detecting the at least one Volatile organic compound;
b. a processing unit configured for:
   i. detecting at least one increase from an initial level of the concentration of the at least one volatile organic compound;
   ii. detecting a corresponding maximum level of the concentration from the initial level;
   iii. detect the return of the concentration of the volatile organic compound towards the initial level or a steady state level (VOCss) from the corresponding maximum level; and
   iv. determining the ventilation rate (k) based on the rate at which the concentration of Volatile organic compound returns towards the initial level or the steady state level from the corresponding maximum level.

In yet further aspects of the present invention, there is provided a system comprising:
a. a compound of interest sensor for providing:
   i. a signal indicative of a first concentration of the compound of interest inside the room, wherein the compound of interest is being released by the person
   ii. a signal indicative of a second concentration of the compound of interest outside the room;
b. the apparatus for estimating a ventilation rate in the room; and
c. the device for estimating the release rate of a compound of interest.

In yet further aspects of the present invention, there is provided a corresponding computer program which comprises program code means for causing a computer to perform the steps of the methods disclosed herein when said computer program is carried out on a computer as well as a non-transitory computer-readable recording medium that stores therein a computer program product, which, when executed by a processor, causes the method disclosed herein to be performed.

It shall be understood that the apparatus/device/system/computer program product claims will have similar advantages as the method claims.

Preferred embodiments of the invention are defined in the dependent claims. It shall be understood that the claimed method, system, computer program and medium can have similar and/or identical preferred embodiments/advantage(s) as the claimed method, in particular as defined in the dependent claims and as disclosed herein.

According to a further embodiment, the Volatile Organic Compound (VOC) is released by a person. Release can be in form of flatulence or other physiological processes. According to yet further embodiment, the VOC can be artificially released, such as by releasing perfume from a perfume bottle.

According to a further embodiment, detecting at least one increase from an initial level of the concentration comprises detecting if the increase is rapid, wherein the detecting if the increase is rapid comprises detecting if the increase in concentration is more than a predefined concentration threshold within a pre-defined time threshold.

Advantageously, this helps to understand certain compounds in the room that are temporary in nature, which essentially means that they will also diffuse in sometime and hence understanding the sudden increase and later rate at which they decease help in understanding the ventilation rate of the room.

According to a further embodiment, detecting a corresponding maximum level of the concentration further comprises extracting a peak from the signal indicative of the corresponding maximum level.

According to a further embodiment, determining the ventilation rate comprises extracting a decreasing phase of the signal from the corresponding maximum level to the initial level.

According to a further embodiment, the method further comprises performing the steps of the method over a period of time and averaging the ventilation rate calculated each time.

According to a further embodiment, the release rate is at least one of an exhalation rate, a flatulence rate and rate of release of the compound from skin.

According to a further embodiment, health of the person is analyzed based on the release rate of compound of interest.

The invention can be integrated in solutions related to parenting and child care, e.g., to determine the excretion of compounds indicative of the health or development of a baby. The invention could be integrated in solutions from the sleep monitoring to analyze the health status of a person while the person is sleeping. The invention could be used for home monitoring purposes to track disease/recovery progression of patients or people at risk of certain health conditions. For instance, researchers have identified certain compounds in the breath related to lung and breast cancer (Volatile biomarkers in the breath of women with breast cancer. Phillips M, Cataneo R N, Saunders C, Hope P, Schmitt P, Wai J, J Breath Res. 2010 June; 4(2):026003). Another example is of diabetes in which acetone levels are elevated due to rise of blood sugar level and intensive lipolysis (On the mammalian acetone metabolism: from chemistry to clinical implications. Kalapos M P, Biochim Biophys Acta. 2003 May 2; 1621(2):122-39). In another example, dysregulation in CO and its levels in exhaled breath has also been implicated in heart disease (Relaxant effects of carbon monoxide compared with nitric oxide in pulmonary and systemic vessels of newborn piglets. Villamor E, Pérez-Vizcaíno F, Cogolludo A L, Conde-Oviedo J, Zaragozá-Arnáez F, López-López JG, Tamargo J Pediatr Res. 2000 October; 48(4):546-53.) The invention could perhaps also be used to detect human emitted compounds of interest indicative of poor mouth/teeth hygiene.

In yet further embodiment of the invention the determined ventilation rate can be further used to control an air purifier or air ventilation system. For instance, if it is determined that ventilation rate is a below a predetermined ventilation rate, then the air ventilation system can accordingly be triggered to increase ventilation. Similarity, if the air ventilation is determined below a pre-determined ventilation rate, then the air purification system can be accordingly triggered.

### BRIEF DESCRIPTION OF THE DRAWINGS

These and other aspects of the invention will be apparent from and elucidated with reference to the embodiment(s) described hereinafter. In the following drawings

FIG. 1 shows an overview of various compound of interest (X) present in human breath;

FIG. 2 shows a typical set up a room in which the person, such as a baby, is monitored;

FIG. 3 shows a flowchart detailing the method steps for estimating a ventilation rate in a room;

FIG. 4a shows an example of a VOC signal observed overnight in a baby bedroom;

FIG. 4b shows a zoomed in view of a particular peak in the signal of FIG. 4a;

FIG. 5 shows a flowchart detailing the method steps for estimating a release rate of a compound of interest;

FIG. 6 shows a block diagram of an apparatus for estimating a ventilation rate in a room according to an embodiment of the invention;

FIG. 7 shows a block diagram of a device for estimating release rate of a compound of interest according to an embodiment of the invention; and

FIG. 8 shows a block diagram of a system for estimating release rate of a compound of interest according to another embodiment of the invention.

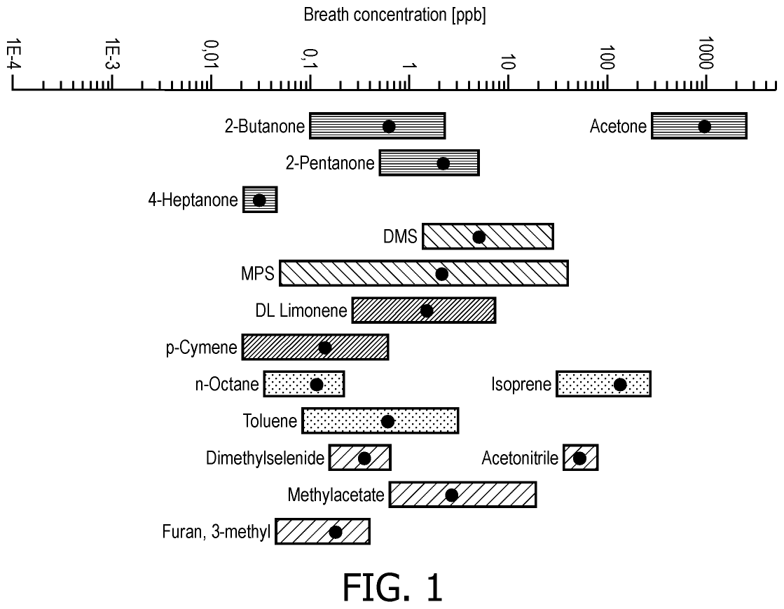

### DETAILED DESCRIPTION OF THE DRAWINGS

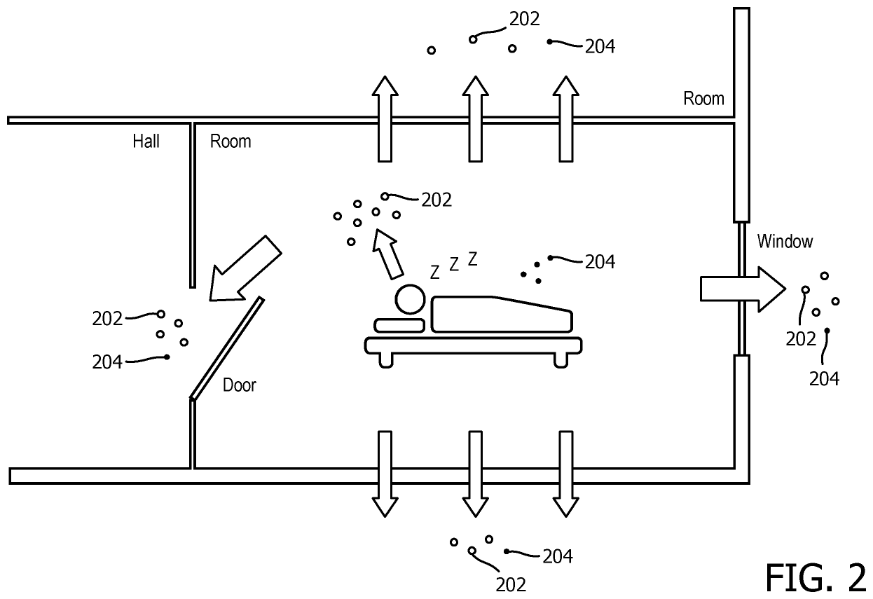

FIG. 2 shows a typical set up a room in which the person is monitored. The person seen in this case is sleeping in the room. It is understood that sleeping is a nice time to monitor a person because there are less disturbances in the room for instance, other people entering the room. To further elaborate, if the health of the baby is to be monitored, then there are high chances that when he/she is sleeping in the night, there will be less interaction with others, such as parents in the room, and hence the concentration of the compounds in the room will be indicative of the compounds released only by him. In the current figure you can see bigger dots 202 are the compounds of the interest, such as CO₂, released by the person by breathing. Further, smaller dots 204 are indicative of Volatile Organic Compounds (VOCs), such as H₂, H₂S, CH₃SH, amines, are released by the person by means of flatulence. As it can be seen that both these compounds diffuse away through means of ventilation (depicted by arrows in the FIG. 2). Thus, it becomes important to understand the ventilation rate of the room in order to understand the release rate (in this case the exhalation rate by means of breath) of the compound of interest in order to further use it to determine the health status.

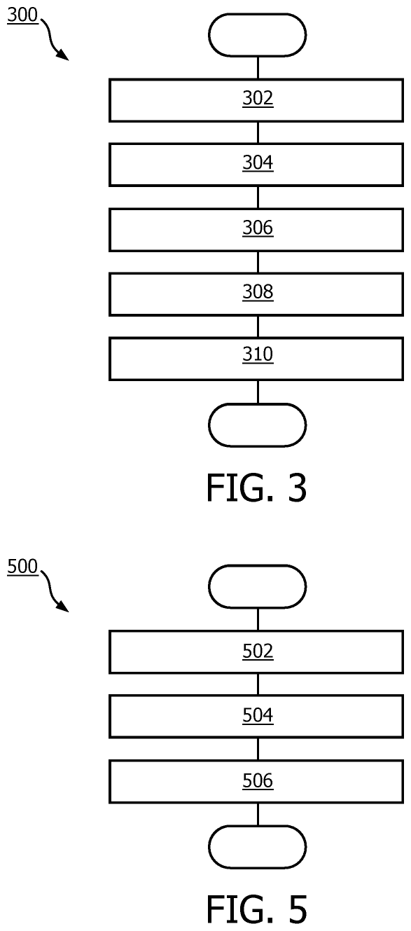

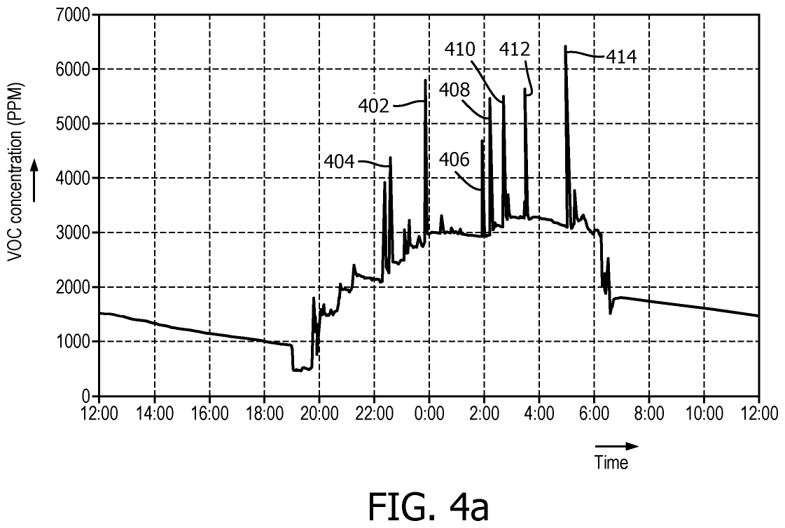

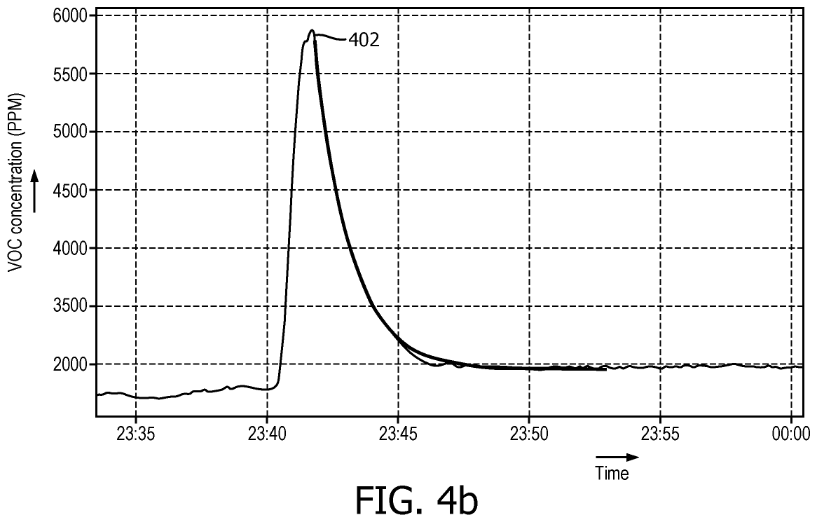

FIG. 3 shows a flow chart (300) detailing the method steps for estimating a ventilation rate in a room. This is explained in conjunction with FIG. 4a and FIG. 4b. FIG. 4a and FIG. 4b show change(s) in concentration of the VOC (y axis) in a time window (x axis). The concentration can be measured/expressed in ppb/PPB (parts per billion) or ppm/PPM (parts per million). The method begins at step 302 by receiving a signal indicative of a concentration of at least one Volatile organic compound (VOC(t)) inside the room. In an embodiment of the invention a VOC sensor can be installed in the room to monitor a VOC. An example of a VOC sensor is a commercially available AppliedSensor iAQ-2000.

At step 304, the signal is processed to detect at least one increase from an initial level (VOC(t0)) of the concentration of the at least one volatile organic compound. It is understood that the typical VOC sensor always detects some or the other VOC in the room. Thus, when there is an increase from a current level, it is indicative of release of a certain VOC in the room. In an embodiment, detecting at least one increase from an initial level of the concentration comprises detecting if the increase is rapid. The rapid increase can be further defined as if the increase in concentration of a VOC is more than a predefined concentration threshold within a pre-defined time threshold.

One such increase in the level is detected and a corresponding maximum level from the initial level (i.e. the peak) is detected at 306. For instance, in FIG. 4a it can be seen there are various increases in the VOC signal, by means of signal peaks (402, 404, 406, . . . , 412, 414), observed overnight (1830h to 0630h) in a room of a baby. Thus, one such peak can be peak 402 can be used and extracted for further determination. In an embodiment, the derivative of the VOC signal can be used to extract VOC peaks, such as peak 402. Many of the temporary increases in VOC used for ventilation determination, are related to events in which instantaneously an amount of gas is released (a step-wise response), such as flatulence. Taking into account the diffusion of the gas inside the room, such increases take in the order of seconds to multiple minutes to reach a maximum.

Thereafter, at 308, it is detected when the concentration of the VOC returns to the initial level or a steady state level (VOC_{SS}), i.e. when the peak 402 comes back to the initial level or the steady state level. This is further depicted in FIG. 4b. In an embodiment, the decreasing part of the peak is extracted. Also the derivative can be used for this purpose. The decreasing part of the VOC peak provides information about the room ventilation rate. It may be noted that each of these peaks return to a base level (steady state level) as can be seen in the FIG. 4a and that is due to the ventilation. The faster the VOC level returns to its initial level, the higher the room ventilation rate.

The decrease in the VOC signal, caused by diffusion due to ventilation to the adjacent areas, can be described by the following ordinary differential equation (diffusion model):

$$\frac{d [ VOC ] ( t )}{dt} = - k ( [ VOC ] ( t ) - [ {VOC}_{ss} ] ) .$$

Here, parameter k represents the room ventilation constant, i.e. the ventilation rate, and [VOC_{SS}] is the steady-state level (also may be alternatively referred to as the initial level) the VOC reaches after the decreasing phase.

At step 310, determining the ventilation rate (k) based on the time (t) taken by the concentration of the at least one Volatile organic compound to reach to the initial level from the corresponding maximum level. In an embodiment, solving the above differential equation gives:

[VOC](t)=[VOC_{SS}]−e^{-kt}([VOC_{SS}]−[VOC](t₀))

This function describes an exponential decay from VOC(t₀) to VOC_{SS}. In theory, the concentration will never return back to the steady state value VOC_{SS}. However, in practical terms, the ventilation will bring the concentration back to the same level. For example, the steady state value may be considered to be reached when the size of the peak (i.e. VOC(t₀)−VOC_{SS}) is ten times the current difference with the previous steady state value VOC_{SS}. In other words, the peak that has been encountered has decreased by 90% of its height. This multiplier of 10 may be any other suitable number, such as 5 or 20.

It may instead be considered that the original level has been reached after 2 time constants of the decay function, or after 3 time constants, or after 4 time constants.

The parameter k relates to the time constant of the decay function. Thus, determining or estimating the value of the parameter k is equivalent to determining a rate at which the concentration of the at least one volatile organic compound returns towards the initial level or the steady state value. The rate represented by the parameter k is not a linear rate but an exponential factor.

However, any measure which represents how quickly the increase in concentration is lost as a result of ventilation may be used, and the term “rate at which the concentration returns towards . . . ” should be understood accordingly.

Parameter k is determined/estimated using a least-squares method that minimizes the sum of squared differences between the simulated VOC level and the measured VOC level. To further elaborate, the diffusion model is fitted using a least-squares technique to estimate the room ventilation rate. The black line in FIG. 4b shows the simulated VOC level.

It may be appreciated that the above procedure can be repeated for each other observed peak, i.e. 404 . . . 412, to update the room ventilation rate. Thus, one could also select all identified peaks during a period of time and calculate an average ventilation rate, which will facilitate in a more robust estimation.

FIG. 5 shows a flow chart 500 detailing the method steps for estimating a release rate of a compound of interest, such as compound X. In an embodiment the compound of interest can be CO₂. The method described in the FIG. 5 works in conjunction with FIG. 3. The method begins at step 502 by receiving a signal indicative of a first concentration of the compound of interest ([X](t)) inside the room, wherein the compound of interest is being released by the person. In an embodiment, this information is received from a compound of interest sensor, such as CO₂ sensor to determine CO₂ exhalation. Another example of such a sensor can be a hydrogen sensor. In an embodiment the compound of interest may also be another VOC of interest, such as acetone. In this case the sensor can be an acetone sensor. It may be appreciated that the VOC of interest will be different than the VOC used in FIG. 3 to determine the ventilation rate. The VOC used for determining the ventilation rate are the ones which are temporarily present in the room, for instance by means of flatulence. In an another embodiment, compound of interest sensor can in fact monitor/detect multiple compounds of interest simultaneously.

At step 504, a signal indicative of a second concentration of the compound of interest (Xₒᵤₜ) outside the room is received. In an embodiment, this information is also received from a similar sensor as in the step 502, which is placed adjacent, preferably immediately outside, to the room where the person is located/sleeping.

At 506, the release rate of the compound (h_{excr}) of interest is calculated based on the first concentration, second concentration and the ventilation rate. This is further elaborated below.

In the current embodiment, the release rate is an exhalation rate by means of breathing. A computational model similar to the one used in the method of FIG. 3 is used to reproduce/simulate the measured dynamics of compound X. The model consists of two parts: a part that describes the human exhalation/excretion of X and a part that describes the diffusion of X to adjacent areas making use of the previously estimated room ventilation. The model is given by the following ordinary differential equation that describes the change in the concentration of X over time:

$$\frac{d [ X ] ( t )}{dt} = {h}_{excr} - k ( [ X ] ( t ) - [ {X}_{out} ] ) .$$

Here, h_{excr} represents the human release rate, also may be referred to as human excretion rate of the compound, [X] is the room concentration of the compound, [Xₒᵤₜ] is the outside concentration of the compound (which in many cases is probably negligible small), and k is the room ventilation rate estimated by means of method steps of FIG. 3. Solving the differential equation gives:

$$[ X ] ( t ) = \frac{1}{k} ( {h}_{excr} + k [ {X}_{out} ] - {e}^{- kt} ( {h}_{excr} + k [ {X}_{out} ] - k [ X ] ( {t}_{0} ) ) )$$

The parameter of interest h_{excr} is estimated/calculated using a least-squares method that minimizes the sum of squared differences between the simulated compound level and the measured compound level.

In various embodiment of the invention, once the exhalation rate is determined, which essentially means the exhalation rate of a particular compound of interest, it can be further used to analyze health of the person based on this information. For instance, the compound of interest can be Acetone and Acetone is also known to be elevated in diabetic patients. Thus, understanding the release rate of Acetone can provide insightful analysis of condition of the patient unobtrusively.

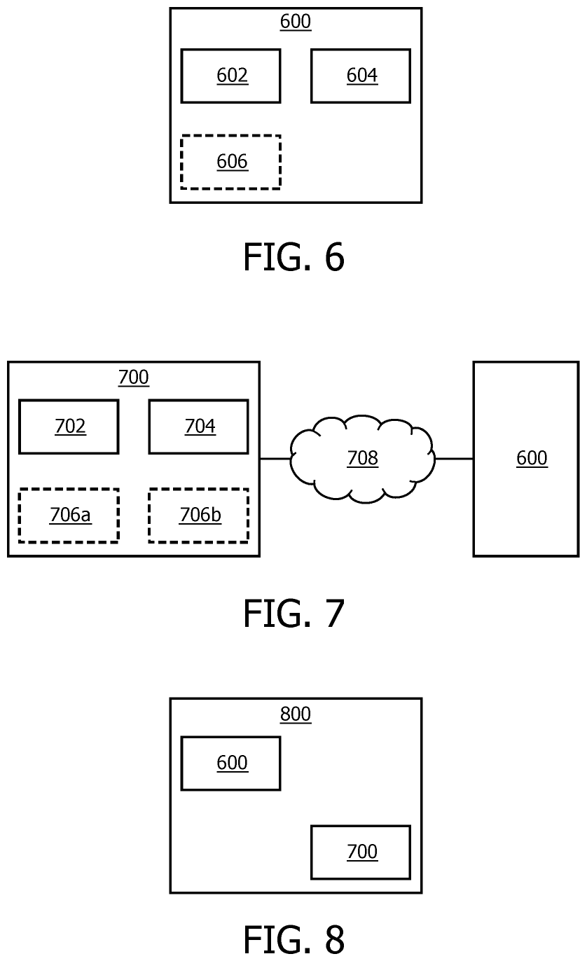

FIG. 6 shows a block diagram of an apparatus 600 for estimating a ventilation rate in a room according to an embodiment of the invention. The apparatus 600 includes a signal interface 602 for receiving a signal indicative of a concentration of at least one Volatile organic compound inside the room, wherein the signal is received from a sensor 606 configured for detecting the at least one Volatile organic compound. In an embodiment of the invention this sensor can be located either within, i.e. integrated in, the apparatus 600 or outside the apparatus 600 (hence depicted by dotted lines) communicating either wirelessly or in a wired manner with the with the signal interface 602. The apparatus 600 further includes a processing unit 604 to determine ventilation rate of the room. The processing unit 604 process the signal by performing the steps 304 to 310 as explained in FIG. 3.

FIG. 7 shows a block diagram of a device 700 for estimating release rate of a compound of interest according to an embodiment of the invention. The device 700 includes a Compound of interest signal interface module 702 for receiving a signal indicative of a first concentration of the compound of interest inside the room, wherein the compound of interest is being released by the person; and receiving a signal indicative of a second concentration of the compound of interest outside the room. In an embodiment of the invention, the signal indicative of the first concentration and second concentration is received from a first sensor 706a placed inside the room and a second sensor 706b placed outside the room. These sensors are of the same type since they are measuring the same compound. These sensors can be part of the same device 700 or can interact either wireless or in the wired manner with the Compound of interest signal interface module 702. In another embodiment, these sensors can also monitor more than one compound of interest. Once the both signals are received, a processing unit 704 then calculates the release rate (h_{excr}) of the compound of interest based on the first concentration, second concentration and the ventilation rate of the room. The calculation of the release rate of the compound of interest has been explained in detail in conjunction with FIG. 5. The ventilation rate required by the device 700 to calculate the release rate (h_{excr}) of the compound of interest is provided by the apparatus 600. In an embodiment of the invention, the device 700 can be in an electronic communication with apparatus 600. The communication can for instance be wired or wireless, such as network 708. In another embodiment of the invention, the apparatus 600 can be part of the device 700, which is also depicted by FIG. 8, where each of the apparatus 600 and device 700 form sub-modules of a system 800.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

A computer program may be stored/distributed on a suitable non-transitory medium, such as an optical storage medium or a solid-state medium supplied together with or as part of other hardware, but may also be distributed in other forms, such as via the Internet or other wired or wireless telecommunication systems.

Any reference signs in the claims should not be construed as limiting the scope.

1. A method of estimating a ventilation rate in a room, the method comprising:
receiving a signal indicative of a concentration of at least one Volatile organic compound (VOC(t)) inside the room, wherein the compound is released by at least one of exhalation, flatulence and release of the compound from the skin of a person in the room;
processing the signal to
detect at least one increase from an initial level (VOC(t0)) of the concentration of the at least one volatile organic compound;
detect a corresponding maximum level of the concentration from the initial level;
detect the return of the concentration of the volatile organic compound towards the initial level or a steady state level (VOCss) from the corresponding maximum level; and
determine the ventilation rate (k) based on the rate at which the concentration of the at least one Volatile organic compound returns towards the initial level or the steady state level (VOCss) from the corresponding maximum level.

2. The method according to claim 1, wherein detecting at least one increase from an initial level of the concentration comprises detecting if the increase is rapid, wherein the detecting if the increase is rapid comprises detecting if the increase in concentration is more than a predefined concentration threshold within a pre-defined time threshold.

3. The method according to claim 1, wherein detecting the corresponding maximum level of the concentration further comprises extracting a peak from the signal indicative of the corresponding maximum level.

4. The method according to claim 1, wherein determining the ventilation rate comprises extracting a decreasing phase of the signal from the corresponding maximum level to the initial level.

5. The method according to claim 1, wherein the method further comprises performing the steps over a period of time and averaging the ventilation rates calculated.

6. The method according to claim 1, further comprising estimating a release rate of the compound from the person, wherein estimating the release rate further comprises:
a. receiving a signal indicative of a first concentration of the compound of interest ([X](t)) inside the room, wherein the compound of interest is being released by the person;
b. receiving a signal indicative of a second concentration of the compound of interest (Xₒᵤₜ) outside the room; and
c. calculating the release rate of the compound (h_{excr}) of interest based on the first concentration, second concentration and the ventilation rate.

7. The method according to claim 6 further comprising analyzing health of the person based on the release rate of compound of interest.

8. The method according to claim 1, wherein the person is a baby.

9. An apparatus for estimating a ventilation rate in a room, the apparatus comprising:
a. a signal interface configured for receiving a signal indicative of a concentration of at least one Volatile organic compound inside the room, wherein the compound is released by at least one of exhalation, flatulence and release of the compound from the skin of a person in the room and wherein the signal is received from a sensor configured for detecting the at least one Volatile organic compound;
b. a processing unit configured for:
i. detecting at least one increase from an initial level of the concentration of the at least one volatile organic compound;
ii. detecting a corresponding maximum level of the concentration from the initial level;
iii. detecting the return of the concentration of the volatile organic compound towards the initial level or a steady state level (VOCss) from the corresponding maximum level; and
iv. determining the ventilation rate (k) based on the rate at which the concentration of Volatile organic compound returns towards the initial level of the steady state level (VOCss) from the corresponding maximum level.

10. The apparatus according to claim 9 further comprising a Volatile Organic Compound sensor for providing the signal indicative of a concentration of at least one Volatile organic compound inside a room.

11. A system comprising:
a. a first compound of interest sensor for providing a signal indicative of a first concentration of the compound of interest inside the room, wherein the compound of interest is being released by a person and is released by at least one of exhalation, flatulence and release of the compound from the skin of the person;
b. a second compound of interest sensor for providing a signal indicative of a second concentration of the compound of interest outside the room;
c. an apparatus for estimating a ventilation rate in a room according to claim 10; and
d. a device for estimating release rate of the compound of interest, wherein the release rate is of the person, comprising a processing unit configured for calculating the release rate (h_{excr}) of the compound of interest based on the first concentration, second concentration and the ventilation rate provided by the apparatus.

12. A computer program product comprising computer program code which, when executed by a computer is adapted to receive a signal indicative of a concentration of at least one Volatile organic compound inside a room and to perform the steps of processing of the method according to claim 1.
